(19) United States
(12) Patent Application Publication
(10) Pub. No.: US 2015/0223906 A1
(43) Pub. Date: Aug. 13, 2015

(54) Medical Procedure Localizing Aid

(71) Applicant: Target Tape Inc., Vancouver (CA)

(72) Inventors: Colin Stephen O'Neill, West Vancouver (CA); Nicholas Drake Seto, Vancouver (CA)

(21) Appl. No.: 14/422,737

(22) PCT Filed: Aug. 27, 2013
(86) PCT No.: PCT/CA2013/000751

§ 371 Date: Feb. 20, 2015

Related U.S. Application Data
(60) Provisional application No. 61/743,031, filed on Aug. 27, 2012.

(51) Int. Cl. A61B 19/00

(52) U.S. Cl. CPC A61B 19/54; A61B 2019/5491; A61B 2019/5466; A61B 2019/5454

(57) ABSTRACT

A medical procedure localization aid produces reference marks on both the patient and the medical imaging scan. The aid is defined by an imaging substrate having indicia on one side that is opaque to medical imaging and a second substrate having indicia that appears on the patient's surface for the procedure. Anchor points align the second substrate to the same position as the first substrate on the patient. By visualizing the location of a target on the scan image relative to the indicia on the scan image and comparing that with indicia on the patient, a medical processional may reliably locate where a medical procedure should be performed.

### TECHNICAL FIELD

This invention relates to apparatus and methods used in the medical field, and more specifically to apparatus and methods for medical procedure localization.

### BACKGROUND

A medical imaging scan, such as an X-ray, computerized tomography, position emission tomography, and/or magnetic resonance imagining, is commonly utilized first to determine if a surgical procedure is necessary. The medical professional relies on this image created by the imaging scan to view the precise internal information of the patient and to assist in confirming a diagnosis. Medical aging is typically performed at two different times: preoperatively and intraoperatively. Preoperative imaging occurs outside of the operating room, while intraoperative imaging is used in the operating room during a procedure.

A preoperative medical image is generally taken for all patients before a procedure or surgery, and as noted it is usually needed to confirm a diagnosis. When the preoperative medical image is performed for diagnostic purposes, there is a chance that the patient would not need any further medical intervention. The preoperative medical imaging scan may happen the day of the procedure or many months in advance, and the imaging may occur at different facilities with different people. It is common that the radiologist performing the scan has the full patient history, which would allow them to ascertain the probability that a procedure will be required. When a procedure does occur at a later time and/or in a different facility, the medical professional who will perform the procedure may not have been assigned to the patient until after the preoperative scan. If the medical image shows irregularities, the medical professional may then conclude that a medical procedure is necessary. The medical professional could use this medical image to localize the site of the procedure. Visible and palpable landmarks relative to the target may be referenced on the scan to determine the location of the surface skin site. Examples of these various procedures include, but are not limited to: surgical incisions to treat fractures, spinal and thoracic lesions, the removal of foreign bodies, and biopsies. These medical procedures are not limited to human subjects, but can also include other animals or cadavers for veterinary or clinical research procedures.

The medical professional usually relies on the initial preoperative medical image for guidance to the target location. If the initial preoperative image is not sufficient, intraoperative imaging techniques, such as fluoroscopy or ultrasound, are commonly utilized. Intraoperative imaging provides sequential, real-time images.

Both preoperative and intraoperative medical images will show the internal point of interest. However, the medical image will show no visual of the patient's external anatomy, which would be useful to localize exactly where the procedure should be performed. If intraoperative imaging is not utilized, the medical professional may rely on palpation of anatomical landmarks to try and perform their procedure directly above their targeted area of interest. This method is prone to inaccuracy due to the difficulty of feeling for landmarks and often requires an extension of the incision or working awkwardly through an angled trajectory. In some instances, the medical professional may even accidentally perform the procedure on the wrong area because the site of the incision was inaccurate. Landmarks, such as joints, may also be visible. The medical professional may have difficulty eye-bailing the distances from the landmark to the target. Variability in patient characteristics, such as atypical anatomy, compounds the difficulties of site localization. Certain regions of the body, like the spine, have repetitive structures that further increase the difficulty of determining the target region.

Intraoperative imaging techniques, such as fluoroscopy or ultrasound, may be on-hand for the entire procedure and can be used for initial localization. However, using these technologies for surface localization may be overly complex and cumbersome. For example, the medical professional may need to hold a metallic surgical tool in the field of view of the X-ray on the surface of the skin to act as a reference point. Repeated images may be taken with the metallic tool being placed in different positions until it aligns directly over the target, increasing the time of the procedure. Intraoperative fluoroscopy also exposes the medical professional, operating room staff and patient to added radiation. When the medical professional has to use a metal instrument, they are inclined to use their hands to hold it in place during imaging, causing direct exposure that is the most dangerous to them. In many cases the target site on the surface of the patient is in a two-dimensional space, requiring alignment in both directions for surface site locations. Moreover, intraoperative imaging modalities can be less clear and detailed versus preoperative imaging such as MRI and CT. The target in the patient that is visible within the detailed preoperative scan may not be visible on the intraoperative scan, such as very fine bone fractures or lesions in non-bony flesh. Disk abnormalities in spinal procedures are rarely visible in intraoperative-based imaging. These intraoperative radiograph machines can be quite costly too, or just not worth the burden, with medical professionals performing procedures based off preoperative imaging alone.

When a patient enters the operating room, the medical professional may first use a skin marker to sketch out their incision. As discussed above, they may use a medical image, palpation and/or eye-balling to determine the operating site. The patients skin is cleaned, usually with a compound like ChloraPrep™. An incise drape may also be applied for infection control. The medical professional would then make their incision. Multiple sites may be required for a procedure, requiring an iteration of the localization techniques to mark out each site. It should be noted that it may not be an incision, but any other process used in a medical procedure, such as punctures from catheters and needles.

As might be expected, inventors have created several types of aids to assist with medical imaging guidance. U.S. Pat. No. 6,333,970 to LeMaitre et al (2001) discloses an adhesive with radiopaque indicia in the form of a linear graduated pattern. The adhesive is placed on the patient before the scan and items underneath the skin can be sized and their location determined. However, when the patient enters the operating room, the adhesive is removed along with the reference marks on the body. The locational marks on the scan are not useful if they can no longer be referenced to on the body. Therefore, LeMatire's tape is primarily used to aid diagnostics of an X-ray image.

U.S. Pat. No. 4,506,676 to Duska (1985) utilizes a radiopaque dotted line on an adhesive tape that will guide the medical professional to the area of interest on the X-ray image. This device will show as a line on the X-ray image, but does not provide direct locational guidance on the body when the patient enters an operating room.

U.S. Pat. No. 5,848,125 to Arnett (1998) also attempts to bring locational information to an X-ray image by placing a small metallic pellet underneath an adhesive. The pellet gives doctors a reference point to look for on the X-ray image, where they then note and estimate the landmark closest to the pellet. When it comes time to perform the procedure, they will again estimate the distance from that landmark to the area of interest. The pellet also obstructs the medical professional from marking the skin of the incision site with their surgical pen. The pellets must be removed, and thus rendered useless, if the medical professional marks their area of interest.

U.S. Pat. No. 5,193,106 to DeSena (1993) discloses radiopaque stickers with flat shapes formed thereon including a circle, the outline of a square, and the outline of a triangle. This device is limited to small shapes for the foot and must be removed before the procedure.

Inventors have also proposed devices to aid in making more accurate incisions. U.S. Pat. No. 6,972,022 to Griffin (2005) discloses a skin-marking device that marks skin with a radiopaque substance, a fluorescent composition, a non-magnetic hydrogel for magnetic resonance imaging, a sterilizable gel ink, a combination of any of these, and a mixture of any of these. Using a free hand pen to mark the skin for locational purposes is messy, limits the precision of designs and the accuracy of consistent spacing. This method is time consuming and does not provide the ability to create standard guides for the technicians performing the medical scans. The marks on the skin would also be opaque to follow up medical imaging scans. The medical professional will have to remove the markings if a clean scan is needed, thus rendering the pen markings useless. The material may also need to be removed before performing a procedure due to sterility concerns and biocompatibility with open incisions.

U.S. Pat. No. 5,323,452 to Russell et al. (1999) discloses an alternate marker system for radiography which includes an elongated base tape, a bendable, fabric covered wire containing a material that is radiopaque, and a continuous row of adhesive pads fixedly aligned along the wire. The adhesive pads and the carried radiopaque wire are manually removable from the base tape together with the wire for releasable adherence to a subject. When imaged, the wire will show up as a continuous line. However, if the line is far away from the target of interest, localizing it on the skin will be prone to inaccuracies. If the line is over top an area of interest, the medical professional will still have to visually estimate where along the line the target is since there are no reference marks along the line. The wire would also have to be removed before the patient enters the operating room or before the medical professional begins performing the procedure.

U.S. Pat. No. 7,677,801 to Pakzaban (2010) discloses a device that utilizes a cross-hair projected on a patient's back by lasers. Attached to the device are radiopaque cables to assist with targeting the correct vertebrae. This device is expensive and must be sterilized before every use. It is also time consuming. If the device is placed over top of the incorrect vertebrae, it must then be moved up or down the patient's back and a subsequent scan must be taken. This process must be repeated until the medical professional has the laser cross-hair directly over the targeted vertebra.

Other inventors have proposed aids to assist with locational guidance for inserting biopsy needles into a patient's body. U.S. Pat. No. 4,860,331 to Williams et al. (1989) discloses an adhesive tape structure with a plurality of radiopaque vertical lines, with biopsy needle holes formed between the parallel vertical lines. This structure is said to be useful during computerized tomography scans to aid in locating the appropriate position to insert a biopsy needle. This device cannot be removed because there will be no reference for the medical professional to know where to insert their needle. The accuracy of this device is limited because the medical professional is restricted to inserting the needle only through the holes in the device. The hole may or may not be directly above the optimal entry point for biopsy needle insertion.

U.S. Pat. No. 6,714,628 to Broyles et al. (2004) expands upon the '331 patent of Wiliam et al., described above, with an adhesive with a plurality of radiopaque vertical lines, with vertical cutouts between the radiopaque lines. This device gives a larger area to insert the biopsy needle into, but still leaves a chance that the cut out area is not directly above the optimal entry point for biopsy needle insertion. This device has limited practical surgical use and an inconvenient method of imprinting any reference marks on the body.

U.S. Pat. No. 5,052,035 to Krupnick (1991) is similar to '628 and '331, as it also describes a radiopaque pattern printed on a single substrate. However, in this case the pattern is defined as a 2D grid, not a 1D ruler. The substrate may be porous or have small cut-outs to allow for the demarcation of the skin underneath. Adhesive on the device is layered outside the grid region so as to not obstruct the marking of the skin. This patch is designed to be used over a short period of time during an image-guided procedure; therefore, the bond of the patch is not strong (selectively applied). On the off-chance that this patch is dislodged from the skin during the procedure, there are two reference point holes through the patch where the practitioner can mark out two skin dots at the start. The practitioner can then align those two skin points with the reference point holes on the dislodged patch to re-place the patch to its initial position. The device is designed for use with imaging in cases where the scan and procedure take place in the same timeframe and location. The device is not designed for universal applications, such as for a procedure that uses preoperative imaging and a later surgery. The patch could not stay on the body for extended periods of time to allow a medical professional to later localize their target. In addition, the single-layer nature of the device does not allow for a dual optimization in both an imaging and a later operative environment.

Inventors have created several types of aids to assist with medical imaging guidance. U.S. Pat. No. 7,853,311 to Webb (2010) describes a targeting device comprised of a series of radiopaque coordinates/lines within a sterile, non-porous, flexible surgical drape that can be adhered to the skin. Once the entry point has been determined, the medical professional cuts through the device. There are a variety of disadvantages of said device for certain procedures. First, any follow-up scans will continue to have the radiopaque indicia appear on the image while the device remains adhered to the skin. It may be desired to remove the targeting system because subsequent scan images may not require the radiopaque indicia. The radiopaque indicia would obscure the image or act as a distraction to the medical professional. Confirmation images may also need to be saved mid-procedure to confirm the placement of screws or other hardware where the radiopaque indicia would need to be removed to ensure there is no obstruction. However, removal of the device will render the antimicrobial purpose of the device useless. Moreover, '311 does not specify how the medical professional can cut through a radiopaque indicia. If the indicia is made of metal or a radiopaque-paste, the scalpel or other instrument may have difficulty entering into the skin. There are also risks of radiopaque material being displaced from the drape, like flaking off if it is a paste, and potentially entering the wound. In addition, the adhesive of the drape makes it difficult to move once placed; and once removed, the drape is very elastic and easily crinkles and adheres onto itself. The medical professional may wish to orient the grid differently after their first image, for example if it is desired to be in parallel to an internal structure, or if the grid misses the target location altogether. Moreover, the device was created and works only for intraoperative imaging. After the device has been used in a preoperative medical imaging it would have to be removed before an incision because the device was placed in a non-sterile environment. This would make it impossible for the medical professional to use the device to correlate with the preoperative medical scan to make an incision. Overall, there are a variety of shortcomings of the claimed device and methods in '311 that restrict it from being an easy-to-use, universal localization aid.

U.S. Pat. No. 8,195,272 to Piferi (2012) describes an MRI-compatible patch for identifying a surface location. There is material opaque to the MRI on the top layer in a grid pattern. The top layer can release from a bottom layer that has the same grid pattern printed on it; the bottom layer allows for the user to mark the target site. The device is used in conjunction with an intraoperative brain procedure in which a computer and an MRI machine is used to guide an instrument to a specific region in the brain. This MRI patch can be read by a computer algorithm, which then outputs a surface coordinate that can be found on the bottom layer, which the medical professional uses to make their skull bore hole. As described, the device can only be used during a medical procedure with MRI. The top layer opaque indicia can be very detailed, allowing for the computer to recognize the grid. The obstructive top layer can then be removed to allow for the skin site marking. The bottom layer then is only for referencing, to mark the target site on the patient's skin. For example, the bottom layer, with its printed coordinates, may have perforations that allow for regions of it to be removed, exposing the skin for demarcation of the site.

US Patent publication No. US20120302863 A1 to O'Neill, which is assigned to the assignee of the present application and is commonly owned by the same entity, describes a targeting device that has a substrate with an upper surface with radiopaque indicia patterned as a locational guide (ex. grid) and a lower surface with patient marking indicia. The patient marking indicia is a transfer of material that creates a visual image on the patient in the same pattern as the radiopaque indicia. This allows the medical professional to correlate between the medical scan and the patient's skin. The transfer material is skin ink. In essence the device is stamping the locational guide pattern on the skin after a medical image is taken. The transfer mechanism would allow the device to be used in limited preoperative imaging or during intraoperative imaging. The disadvantage of the skin ink pattern is it would fade over time making it difficult for the medical professional to correlate with the pattern in the operation. Skin ink marks can rarely last beyond ten days. A significant portion of preoperative imaging occurs over a month before the operation, making this type of device unfeasible to utilize universally.

The patents described briefly above demonstrate that there is a distinct need for apparatus and methods that allow non-invasive and accurate medical procedure localization which a medical professional can use universally to reliably locate a precise target location on a patients body for medical procedure. The imaging and surgical environment would greatly benefit from a versatile device that can be incorporated not only with intraoperative imaging but also with preoperative imaging over various lengths of time. Moreover, a universal device can be optimized to create the most benefits to the medical professional in the various environments.

### SUMMARY OF THE INVENTION

The apparatus and method of the present and illustrated inventions are based on a device that defines an improved means and method of non-invasively locating a procedure site on a patient prior to the medical procedure. The inventive apparatus may be used in connection with numerous types of medical imaging scans, such as, but not limited to: X-rays, computerized tomography, position emission tomography, ultrasound and magnetic resonance imagining. The overall aid will produce reference marks and indicia on both the patient and the medical imaging scan and the indicia on the scan registers with the marks on the patient. The aid is utilized in two phases during which the indicia on the medical imaging scan may be used separately from the reference marks on the patient.

The first phase involves the aid or part of the aid, being attached conformally to the body surface before a medical imaging scan. The resulting image from the scan shows a visual of both the internal anatomy and an opaque locational pattern from the aid itself, herein referred to as the scan locational pattern—the word “opaque” meaning herein a material that would appear on the medical image. The scan locational pattern, which may be a grid, is used to correlate with the internal anatomy and/or specific targets of the patient for the procedure.

The second phase involves the visualization during the procedure of the scan locational pattern on the patient's surface, herein referred to as the skin image. The skin image appears on the patient at the time of the procedure. The pattern of the skin image is similar and corresponds to the scan locational pattern. Moreover, the skin image and the previous scan locational pattern are located at the same place on the patient's surface. Stated another way, the skin image on the patient registers with the scan locational pattern both in terms of position and orientation of the aid on the patient. Therefore, a practitioner is able to accurately correlate the patient's internal anatomy to the scan locational pattern image, which in turn can be correlated to the skin image at the time of the procedure. The skin image allows for the demarcation of the incision site or allows for the procedure to be carried out with the skin image remaining in place.

Anchor points are a system used to connect the first and second phases to ensure that the skin image is placed in the same position and orientation on the patient as the scan locational pattern. During the first phase, markings are made on the patient's skin at designated anchor point sites—the anchor point sites are positioning guides that show the professional where to mark the patient's skin. The part of the aid that creates the skin image on the patient's surface also has the pattern of the anchor points visible on it. The relation between the anchor points with the skin image and the anchor points with the scan locational pattern are the same. Therefore, the anchor points with the skin image can be aligned with and register to the anchor points marked on the patient's surface to place the skin image in the same place as the scan locational pattern. The anchor point pattern must then be asymmetric in nature to eliminate the chance of placing the skin image at an incorrect orientation. This is a key difficulty because a different person could have performed the first phase of the aid, where the initial skin markings may be made at a different time and at a different location. When the locational aid defines a sheet having a symmetric perimeter or periphery, for example, with a rectangular sheet, it would be possible to orient an identical sheet on a patent in a different rotational orientation from the sheet used in the first phase—e.g., 180 degrees rotated relative to the orientation of the first phase. For these and a variety of other reasons, the practitioner in the second phase would not simply know the correct orientation of the skin image so that it accurately registers with the scan locational pattern, even where for instance the positions of the corners of a rectangular sheet used in the first phase were known to the practitioner. Thus, the practitioner may know the proper location for the device, but would not know the correct orientation of the device. Therefore, an asymmetric pattern of anchor points for the skin image is required for proper registration between the skin image and the scan locational pattern. The anchor points thus define aid positioning means that allow for the separation between the imaging phase and the procedure by time and place, while insuring duplication of the position a id orientation of the aid in the second phase, which is important to make the device a universal aid.

In practice, when preoperative imaging occurs with the aid in the first phase, anchor point markings are left on the patient. These reference marks would be maintained on the skin until the procedure. The procedure may be hours, days or weeks later. The device may come with marking pens and/or other tools for the patient to use to maintain the marks on their skin. Importantly, the aid in the first phase and the anchor points on the patient are not uncomfortable for the patient and are largely non-obstructive to other medical processes and normal daily activity. Therefore, if a medical procedure does not end up happening after the imaging, the patient can simply be informed not to maintain the skin markings. Similarly, the aid could still be used in the first phase without the medical professional being connected with the patient yet, allowing the medical professional to later decide if the patient should keep maintaining the markings for the use of the aid in the second phase.

When the procedure is imminent, the second phase occurs. A secondary part of the aid according to the present invention can be aligned with the reference marks on the patient to create the skin image on the patient's surface. Both phases of the aid may occur sequentially in the same time frame. For example, the aid may be used with intraoperative imaging, with the site localization happening immediately afterwards. Due to the complex nature of the relationships between imaging, diagnosis, patients, doctors, surgeons and other medical professionals, the aid needs to be extremely versatile.

The medical professional may reliably use the combination of the medical imaging scan with the skin image that visible on the patient's body to accurately locate the target site for the indicated medical procedure. Accordingly, several objects and advantages of the invention are: to provide visual indicia marks on both the medical image and on the patient's body surface; to provide a quicker and more accurate means of surgical localization; to allow for the greater use of long-term preoperative imaging information during the medical procedure; to reduce the dependence on other intraoperative imaging modalities for localization; to reduce the need of multiple fluoroscopy images and thereby reduce radiation exposure to both patients and medical staff; and to provide optimized location designs which are pre-drawn.

### BRIEF DESCRIPTION OF THE DRAWINGS

For a better understanding of the present invention, in conjunction with other objects, features, and advantages, references should be made to the following description of several preferred embodiments. The preferred embodiments should be read with reference to the appended drawings, in which:

FIG. 1 is an upper perspective and exploded view of a first illustrated embodiment of a medical procedure localization aid according to the present invention, illustrating the five separate layers that comprise the device.

FIGS. 2 and 3 are pictographic flowcharts representing methods according to embodiments of the present invention with an alternative embodiment of a medical procedure localization aid according to the invention.

FIG. 4 is a top perspective view of the embodiment of the medical procedure localizing aid shown in FIG. 2, illustrating the imaging substrate being peeled away from the skin template substrate.

FIG. 5 is a top perspective view of the embodiment of the medical procedure localizing aid shown in FIG. 3, illustrating the imaging substrate being peeled away from the skin template substrate.

FIG. 6 is a schematic view of an exemplary X-ray image of a patient's body (in this image the patients hips and spine) showing the grid lines imprinted on the X-ray from the opaque material deposited on the aid.

FIG. 7 is an X-ray image of the upper torso region of a patient, illustrating the opaque material from a medical procedure localization aid according to the invention as it is visible on the X-ray film.

FIG. 8 is a schematic top view of a first illustrated embodiment of the medical procedure localizing aid according to the present invention positioned on a patient's back with the patient in a prone position.

FIG. 9 is a schematic top view of the upper torso region of a patient shown in a prone position, illustrating the skin template substrate left behind on the patient's body after the imaging substrate is removed.

FIG. 10 is an MR image of the spine region of the patient, illustrating the opaque material from the aid.

FIG. 11 is a top plan view of one embodiment of the imaging substrate layer of a medical procedure localization aid according to the present invention, illustrating one possible indicia pattern, in this case a grid pattern.

FIG. 12 is a bottom plan view of FIG. 11, wherein a separation tab system remains.

FIG. 13 is a top plan view of one embodiment of the skin template substrate layer of a medical procedure localization aid according to the present invention, illustrating one possible skin image pattern, in this case index markings along the perimeter of an open cut-out.

FIG. 14 is a bottom plan view of one embodiment of FIG. 13, wherein a handling tab system remains.

FIG. 15 is a bottom plan view of one embodiment of the protective backing sheet layer of a medical procedure localization aid according to the present invention, illustrating a crack and peel system via two kiss cuts.

FIG. 16 is a top plan view of a further embodiment of the imaging substrate layer of a medical procedure localization aid according to the present invention, illustrating another possible anchor point system.

FIG. 17 is a top plan view of a further embodiment of the skin template substrate layer of a medical procedure localization aid according to the present invention, illustrating a continuous porous or incisable substrate.

FIG. 18 is a top plan view of yet another embodiment of the skin template substrate layer of a medical procedure localization aid according to the present invention, illustrating a substrate with multiple open cut-outs and a full grid pattern as the skin image pattern.

The shading in the drawings is used only to illustrate the invention and is not used to indicate or symbolize any particular material used in the invention.

### DESCRIPTION OF PREFERRED EMBODIMENTS

In a most preferred embodiment, the medical procedure localizing aid according to the present invention is a sterile, flexible adhesive-backed sheet having two primary substrates. As a naming convention, for purposes herein the “top” or “upper” surface/layer of the localization aid is a surface that is exposed, faces away, or is further, from the patient's surface when the localization aid is in place on the patient. The “bottom” or “lower” surface/layer is then the opposite surface—that is, the side facing, or closer to, the patient's surface. The top primary substrate is the imaging substrate. The bottom primary substrate is the skin template substrate. On the bottom of the skin template substrate is a protective cover layer.

It will be appreciated that the apparatus and methods according to the present invention may be utilized by a variety of medical professionals such as doctors, surgeons, radiologists, nurses, technicians, veterinarians and clinical researchers and that the invention is not limited to use by any specific type of professional in any particular field. Moreover, a “patient” for purposes herein may be any subject that a medical professional may perform a procedure on, examples including humans, animals and cadavers.

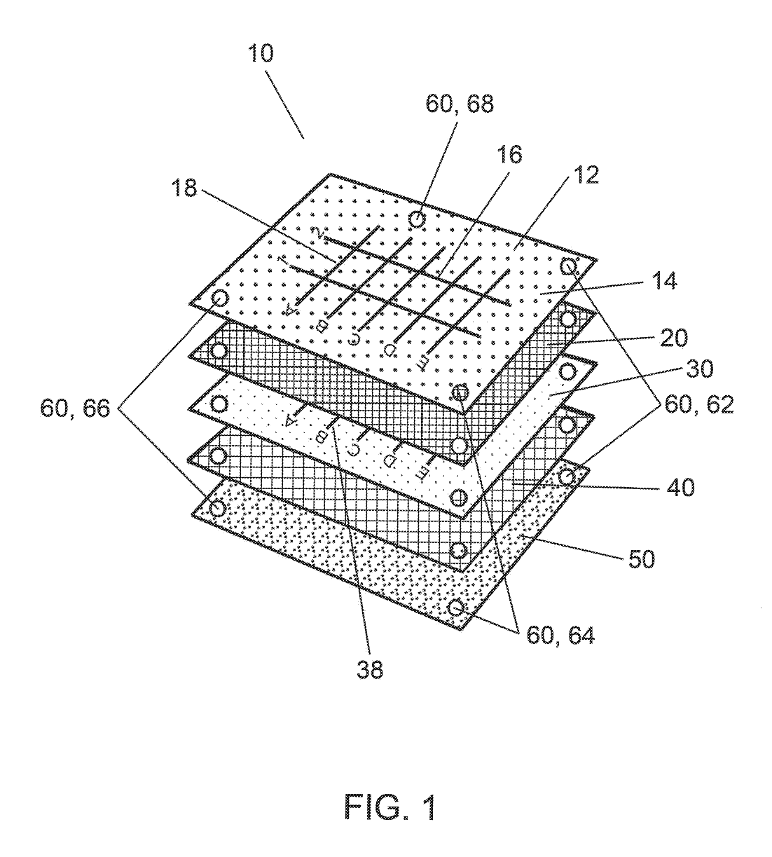

With reference first to FIG. 1, a first illustrated embodiment of a medical procedure localization aid 10 according to the present invention is illustrated in exploded view to illustrate the layers of the device. The five layers of material are laminated and bonded together. The upper or top layer (i.e. the first layer of aid 10) is the layer that is first removed from the patient's surface 84 when the aid 10 is placed on the patient 82 and defines the imaging substrate 12. The upper, exposed surface 14 of imaging substrate 12 includes opaque indicia 16. As noted above, as used herein the word “opaque” means a material that will visually appear on a medical imaging scan. The opaque indicia 16 may be arranged in any effective image on the upper surface 14 and as detailed previously, the opaque indicia visually appears on the medical imaging scan as a scan locational pattern, identified generally with reference number 18.

The next immediately adjacent and second layer is a low strength adhesive coating or layer 20 that is applied to the bottom surface of the imaging substrate 12—the adhesive layer 20 is permanently bonded to the lower surface of imaging substrate 12 over substantially the entire area of the lower surface.

The third layer is immediately adjacent adhesive layer 20 and is the skin template substrate 30. The bond between the adhesive layer 20 and the skin template 30 is non-permanent so that the skin template may be relatively simply separated from the imaging substrate 12. As detailed below, the skin template 30 includes patient marking indicia 38 that conforms either wholly or partially to the scan locational pattern 18 on the imaging substrate 12.

The fourth layer is the skin adhesive 40, which is defined by an adhesive coating that is applied over the entire lower surface of the skin template 30. As with the adhesive coating that defines the adhesive layer 20, the skin adhesive 40 is permanently bonded to the lower surface of the skin template 30.

The fifth or lowermost layer is the removable protective backer 50. The bond between the protective backer 50 and the adhesive that defines the skin adhesive layer 40 is non-permanent so that the protective backer 50 may easily be removed when the aid 10 is ready to be adhered to a patient 82.

Aid 10 includes plural anchor points that are identified generally with reference number 60, and individually in the embodiment of FIG. 1 with reference numbers 62, 64, 66 and 68. The anchor points are defined by holes that extend through each of the five layers described above and shown in FIG. 1. In the ready to use aid 10, the plural holes in the various layers that define the anchor points 60 are thus aligned. In the embodiment of FIG. 1, there are three holes that are positioned near three corners of aid 10 (i.e., holes 62, 64 and 66) and one hole 68 that is staggered along one side of the aid to make an asymmetric anchor point pattern.

During the first phase of use of aid 10 with imaging, the phase when the scan locational pattern 18 is generated, the aid 10 is applied to and adhered to a desired location on the patient's surface 84 (as detailed below, by removal of the removable backing layer 50 to expose the skin adhesive 40 and then applying the aid 10 to the patient 82). When the aid 10 is adhered in the desired location and orientation on the patient 82, skin marks 70 can be made through each hole of the anchor points 60 with an appropriate skin marking pen 80. The anchor points 60 thus define skin mark positioning guides for marking specific locations on the skin. These skin marks 70 are maintained until the time of the procedure during the second phase.

In the second phase, after the first aid 10 has been removed, the anchor points 60 of a second aid 10 is aligned with the skin marks 70 that were made during the first phase and the second aid 10 is adhered to the patient 82 in the same manner as the first aid 10 (as noted, the second phase may occur many days or weeks after the first phase). Because the anchor points 60 in the second aid 10 are in the identical positions as the anchor points 60 in the first aid 10 used in the first phase, the second aid 10 will be located on the patient 82 in precisely the same location as the first aid 10 used during the first phase. As a result, indicia 18 on the second aid 10 will be “registered” with the indicia 18 of the first aid 10. In the second phase, the imaging substrate 12 is then peeled away by separating the imaging substrate 12 from the skin template 30, leaving in place the skin template 30 in a duplicate location and orientation. Because the anchor points are asymmetrically arranged, the second aid 10 that is applied to the patient 82 during the second phase is place in the identical location and orientation as the first aid 10. The asymmetrical pattern of the anchor points 60 insures that the location and orientation of the skin image 32 on the patient 82 (i.e., with the skin template) will be identical to the location and orientation of the scan locational pattern 18 in the medical image.

The actual geometric configuration of the substrates and locational patterns can be a variety of different shapes and sizes. This can range from a large area to cover the chest and back, to small narrow strips for fingers and toes, or any other convenient size or shape. Moreover, it will be appreciated that there are numerous different types of markings that will suffice the criteria for establishing asymmetric anchor points that insure registered placement between the first aid 10 used in the first phase and the second aid 10 used in the second phase. These would include, for example, a randomly configured outer perimeter for the aid, which would be marked on the patient 82 with perimeter tracing, or anchor point holes that have different geometric shapes (i.e., round, square, triangle, etc.).

In addition to the aid 10 illustrated in FIG. 1, there are a variety of alternatives for these basic layers. For example, the adhesive layer 20 is not limited to a physical substance, but may be a heat seal or any other substance or process that has a removable bond between the imaging substrate 12 and the skin template substrate 30. A heat seal will create a low strength, non-permanent bond between the imaging substrate 12 and skin template substrate 30. With continuing reference to FIG. 1 the upper, exposed surface 14 of the imaging substrate 12 can be optimized for the first phase during the medical scan. Indicia that is opaque to medical imaging scans is layered on the imaging substrate 12—as noted above, as used herein the word “opaque” means a material that will visually appear on a medical imaging scan.

A second purpose of the imaging substrate 12 is to act as a support for the second primary substrate, the skin template substrate 30. To assist in the handling of the aid, the imaging substrate 12 can be made of a relatively stiffer material. As later discussed, the skin template substrate may be very flexible, and without a stiffer attachment to act as a support, the skin template substrate may be difficult to hold and manage, as it may easily crinkle and adhere to itself. The imaging substrate still needs to be conformal during the placement of the skin template substrate. A similar mechanism can be seen in 3M's (Minneapolis, Minn.) dual layer Tegaderm-Film™.

The second primary substrate is the skin template substrate, which is below the imaging substrate. The skin template substrate 30 has a top and bottom surface. The top surface has visible patient marking indicia 38 that correlates to and registers with the imaging substrate's locational pattern 18; the correlation between the patient marking indicia 38 and the scan locational pattern 18 may be either complete or partial, as detailed below. The indicia material needs to be biocompatible since the skin template substrate 30 may be present during the procedure and may come in contact with surgical instruments and the patient's surface 84. The bottom surface of the skin template substrate 30 has a skin adhesive 40. The purpose of the skin template substrate 30 is to act as said skin image 32. The skin image 32 is visible to the practitioner and remains in place on the patient's surface 84 via the skin template substrate 30. The skin template substrate 30 is flexible and conformal to appear in a similar position and orientation as the scan locational pattern 18 on the imaging substrate 12. The skin template substrate 30 may be placed hours before the procedure to save on operating room time, and therefore, needs to be durable and breathable like 3M Tegaderm-Film™ The skin template substrate 30 can also be used during the procedure. The skin template substrate 30 must also allow the practitioner to mark out the site of the procedure or carryout the procedure through the device. To achieve this, the skin template substrate 30 may be, but not limited to being: porous, have window cutouts, an open frame, or an incisable material, or a combination thereof.

A removable protective backing sheet 50 is in contact with the skin adhesive 40 to protect, store, and prevent the aid from being adhered inadvertently to itself or other objects.

The imaging substrate 12 is layered over the top of the skin template substrate 30. When the skin template substrate 30 is adhered to the patient's surface 84, the imaging substrate 12 can be removed if the imaging phase is done, leaving behind the skin template substrate 30 on the patient's surface 84. Therefore, the strength of the skin template substrate's 30 skin adhesive 40 must be relatively stronger than the adhesion between the two primary substrates. The adhesion between the two primary substrates may be an ultra-removable adhesive or a heat seal. The angle of peel of the imaging substrate 12 from the skin template substrate 30 also affects the needed strength of the adhesions. This lamination must be strong enough so that during alignment and handling, or removal of the protective backing sheet 50, the two primary substrates do not de-laminate. Tabs on the different substrates and layers can allow for easier handling as well as separation peeling.

Another embodiment combines the imaging substrate 12 and the skin template substrate 30 into one single layered substrate. The combined single layered substrate has an upper and lower surface. The upper surface has the medical scan opaque indicia 16 layered on it in a similar fashion as said imaging substrate 12. The lower surface has a pressure sensitive skin adhesive 40 similar to the lower surface of said skin template substrate 30. The substrate also allows the function of skin marking through the single layered substrate. To achieve this function the single layered substrate may be thin and porous, or it could also have windows cut-out between the opaque indicia 16 to expose the skin when the combined substrate is adhered to the patient 82. A protective backer 50 is layered along the bottom surface of the combined single layered substrate. The combined substrate would have similar asymmetric anchor points 60. The benefit of this embodiment is increased simplicity. However the medical scan opaque indicia 16 may obstruct the practitioner from freely marking the skin or carrying-out a procedure. Moreover, the opaque indicia 16 may require a more stable substrate that is thicker and less conformal, making the device less comfortable for long-term wear during the second phase. In addition, not having a top layer to act as a carrier/holder may make it more difficult to align during the second phase; the combined single layer may want to fold in on itself during placement on the skin. The first two-layered embodiment described above separates the needs of the imaging environment from the needs of the procedure environment to overcome these difficulties.

Another embodiment separates the imaging substrate 12 from the skin template substrate 30 of the first preferred embodiment. In this circumstance they are different parts: a stand-alone imaging substrate 12 and a stand-alone skin template substrate 30. The stand-alone parts may each be disposable or re-usable. If either part is re-usable, a separate skin adhesive tape can be used to keep it placed on the patient 82 during its respective phase. One benefit of separating the parts is decreasing waste. During the first phase of imaging, the skin template substrate 30 is only needed for its skin adhesive 40. During the second phase, the imaging substrate 12 is only needed for its stiffness handling support. However, these stand-alone parts lack the simplicity to act as a universal aid. Separate parts would need to be sourced for the two environments. Moreover, sterilization would be necessary for re-usable parts. The stand-alone imaging substrate 12 would be applied to the patient 82 before the medical image and would also follow the anchor point process. The stand-alone imaging substrate 12 may even be a cut out metal grid. The grid would need designated points for the anchor points 60. The stand-alone skin template substrate 30 may be similar to said disposable skin template substrate. It can be applied close to the procedure time for the medical professional to correlate with and to mark the incision site. The stand-alone skin template substrate 30 may also be re-usable, such as a die-cut polyester. The more re-usable and durable the stand-alone skin template substrate is, the less versatility it has in application. More specifically, if the stand-alone skin template substrate 30 is also a metal grid taped to the patient 82, it could not be applied long before the operation due to wear-ability constraints of the patient 82. It would be difficult to keep a metal grid adhered to the patient 82 for an extended period without discomfort or movement of said grid. In this circumstance the patient marking indicia 38 of the skin image 32 would be opaque to a medical scan and could be removed during the procedure so it is not an obstruction. One benefit of a re-usable stand-alone imaging substrate 12 would be to overcome the uncertainty of whether a medical procedure is going to occur or not; the radiologists can apply the re-usable imaging substrate without consuming a disposable. The skin template substrate 30 may be disposable, with the medical professional being the one to decide to utilize it. The aid 10 may come in kits with other medical devices for the procedure.

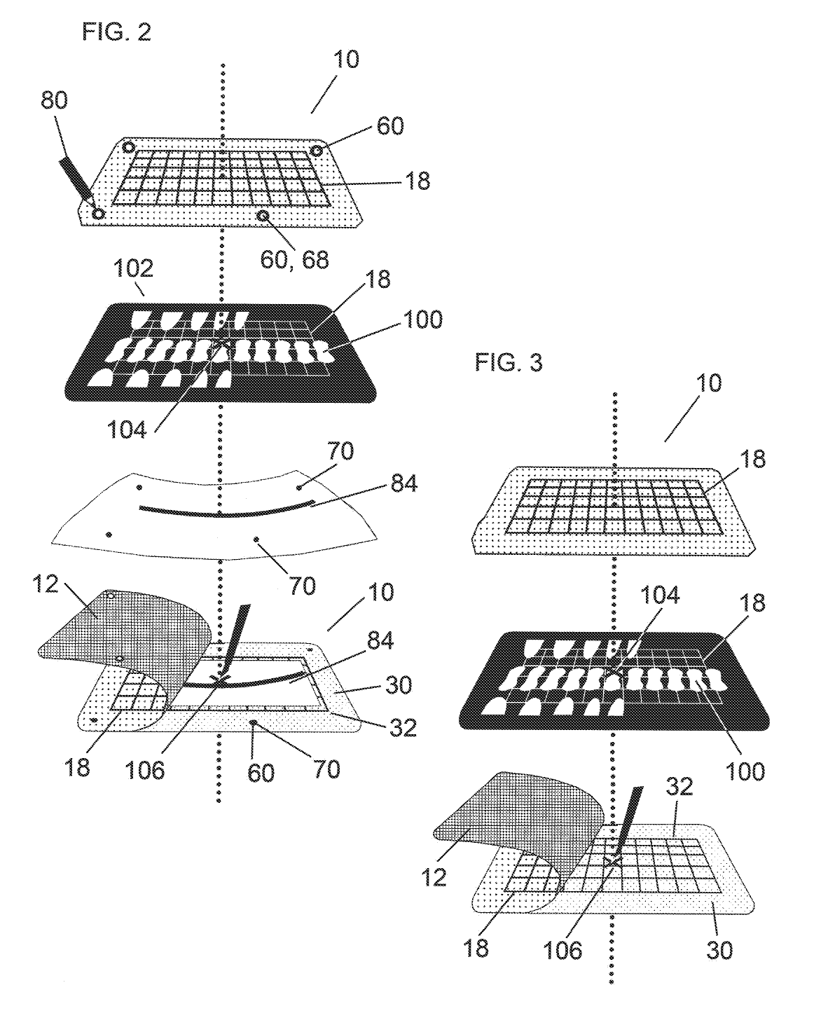

With reference again to the figures, FIG. 2 is a pictographic flow chart outlining a preferred method of use of a preferred embodiment in a preoperative setting. The first step involves the medical professional adhering the localizing aid 10 to the patient 82 over the area of interest. A pen or marker 80 marks the patient's surface 84 through the asymmetrical anchor point holes 60 and creates anchor point skin marks 70 on the patient's surface 84. These anchor point skin marks 70 may be small dots or other shapes marked on the patient's surface 84.

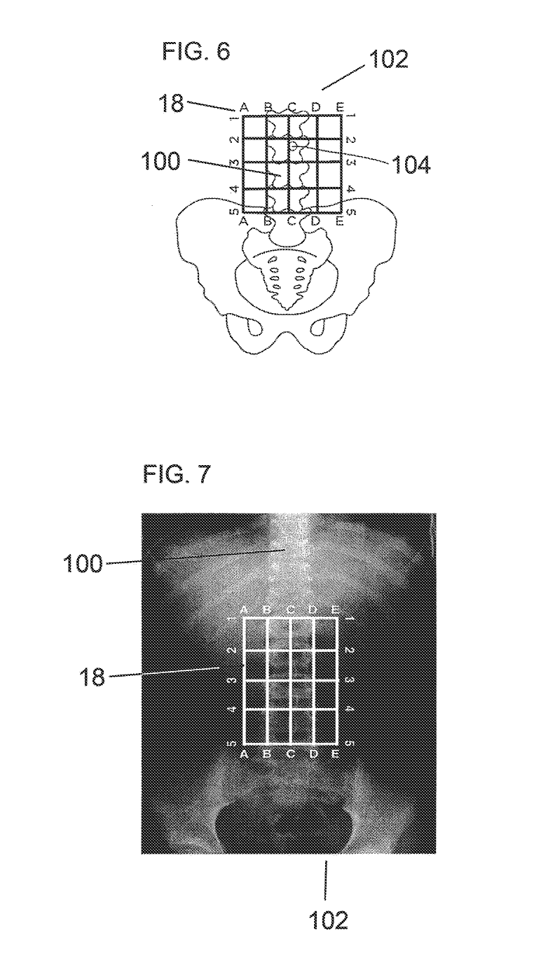

The second step involves the medical imaging. An X-ray, CT, MRI or any other medical imaging scan is performed. The patient's internal anatomy 100, along with the scan locational pattern 18, appears on the image scan 102. FIG. 6 is an AP X-ray image that shows the scan locational pattern 18 in conjunction with the patient's internal anatomy 100, in this example, the spine, pelvis and rib cage. The aid 10 is then removed and disposed.

The third step involves the temporal delay. A time delay between imaging and a procedure can range from several minutes to several weeks. The anchor point skin marks 70 will usually last between 3-7 days if a medical grade skin marker is used. If the time delay is greater, the patient is required to re-mark the anchor point skin marks 70 to prevent fading. In addition, small biocompatible skin films can be used to cover the anchor point markings to reduce the amount of fading. An example skin film used for such purposes is 3M's Tegaderm-Film™, which has been used to preserve the skin markings on patients during prolonged radiation treatment. A package or kit can be provided to the patient that instructs them on how to maintain the dots and could include indelible markers and film protectors. These small anchor point skin marks 70 are more unnoticeable and aesthetic for the patient versus a larger marking. Moreover, it is simpler and easier to maintain smaller anchor point skin markings 70 compared to larger markings or tracing out the entire scan locational pattern.

The fourth step involves correlation during the procedure. Before the incision is made, a second localizing aid 10 is aligned and applied over the anchor point skin marks 70 to duplicate the position of the first device during the imaging step. When the second localizing aid 10 is applied, the top imaging substrate 12 is removed and a bottom layer skin template substrate 30 remains adhered to the patient's surface 84. The second localizing aid 10 can be applied several hours in advance while the patient is in the preoperative room, or it can be applied in the operating room immediately before the incision or other medical procedure processes begin. The medical scan image 102 is referenced and the target 104 is located at its respective coordinate via the scan locational pattern 18. The target's incision spot 106 is then localized and marked by correlating to the skin image 32 via the skin template substrate 30 adhered to the patient's surface 84.

FIG. 3 is a pictographic flow chart showing the preferred method of use of the device in an intraoperative setting. The first step involves the medical professional adhering the localizing aid 10 to the patient 82 over the area of interest.

The second step involves the medical imaging. An X-ray, CT, MRI or any other medical imaging scan is performed. The patient's internal anatomy 100, along with the scan locational pattern 18, appears on the image scan 102.

The third step involves correlation during the procedure. The imaging substrate 12 is removed and the skin template substrate 30 remains adhered to the patient's surface 84. The medical scan image is referenced and the target 104 is located at its respective coordinate. The target's incision spot 106 is then localized by correlating to the skin image 32 via the skin template substrate 30 adhered to the patient's surface 84.

It should be noted that one skilled in the art would be able to modify the preferred methods detailed in FIGS. 2 and 3 to incorporate the single-layered and stand-alone layered embodiments described above into their procedure.

The single-layered, combined embodiment would be adhered to the patient 82 before medical imaging, with the anchor point skin marks 70 on the patient's surface 84. If the imaging were in advance of the procedure, the single layered substrate would then be removed and discarded. Closely before the procedure, a second single layered substrate would be applied to the patient 82 over the anchor point skin marks 70. The medical professional would then mark their incision point(s) 106 through the substrate and then remove it before the medical procedure. Intraoperative usage would bypass the need for anchor point skin marks 70 and utilize only a single device.

The stand-alone embodiment would be utilized in a similar fashion, where the imaging substrate layer 12 is utilized during the imaging phase and the skin template substrate layer 30 is utilized during the procedure phase.

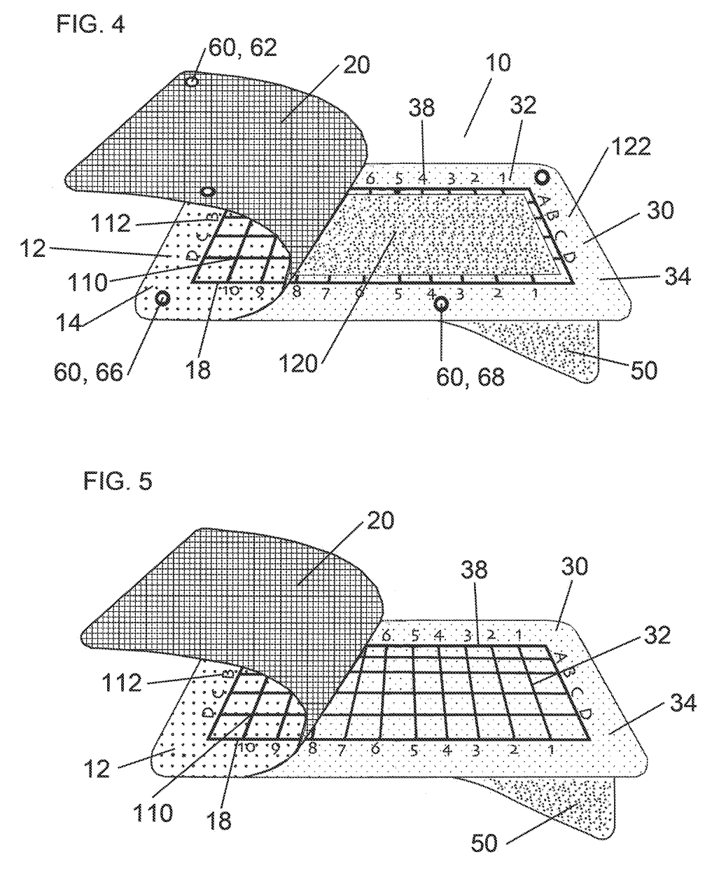

As shown in FIGS. 4 and 5, the preferred embodiment of the localizing aid 10 is comprised of two primary substrates: a top imaging substrate 12 and a bottom skin template substrate 30. The top imaging substrate 12 of the present invention comprises a flexible substrate sheet (i.e. the imaging substrate) 12 having an upper or top surface 14 and opposed lower or bottom surface. The imaging substrate 12 can preferably range from 2-7 mils thick. Example materials include, but are not limited to: PET, Kraft papers and Melinex™. The imaging substrate 12 can be transparent or opaque in color. The top surface 14 includes opaque indicia 16 that will appear visually on the medical imaging scan (See FIGS. 6, 7 and 10) as a scan locational pattern 18. The opaque indicia 16 may be in a grid pattern as shown in FIGS. 6 and 7, which includes vertical and horizontal lines 110 that intersect, or it may take other forms such as dots, cross hatches, circles, graduated linear patterns, and combinations of any of these, or any other logical design that will assist with locating a precise area within a larger space. The scan locational pattern 18 may include reference labels, for example alpha, numeric or other symbols, identified generally in FIGS. 4 and 5 with reference number 112, or other symbols in any other area or direction. It will be appreciated that the combination of vertical and horizontal lines 110 and alpha and numeric symbols 112 are intended to assist the medical professional hi accurately and quickly locating target tissue 106 on the patient's surface 84.

The bottom surface of the imaging substrate 12 includes a low strength adhesive 20, which is applied over the entire surface area of the bottom surface of the imaging substrate 12 to create a non-permanent, light bond between the imaging substrate 12 and the skin template substrate 30. The adhesive used is preferably a biocompatible ultra removable adhesive that results in a low strength bond when it comes into contact with the bottom skin template substrate 30. Alternatively, a heat seal may create the low strength bond between the imaging substrate 12 and skin template substrate 30. One purpose of the imaging substrate 12 is to provide rigidity and support for the bottom skin template substrate 30 to prevent it from flexing and adhering to itself.

The bottom layer of the preferred embodiment, the skin template substrate 30, comprises a flexible, conformal skin film having an upper or top surface 34 and opposed lower or bottom surface 36. The skin template substrate 30 can preferably range from 1-4 mils thick. Example materials include, but are not limited to: polyurethane, polyethylene and polyester. The top surface 34 includes patient marking indicia 38 that will not appear visually on the medical imaging scan 102, but will appear visually on the patient's surface 84 as the skin image 32. The non-opaque patient marking indicia 38 may be in a correlating scan locational pattern 18 that corresponds to the scan locational pattern 18 in a duplicate or partially duplicate pattern. For example, the scan locational pattern 18 may include a full grid with thick lettering that is more visible on medical scans, whereas the skin image 32 pattern may just be perimeter index markings with smaller lettering that is less obstructive to the medical professional on the patient, but provides sufficient details for localizing the correct coordinates. In certain circumstances the patient marking indicia 38 of the skin image 32 could also be opaque to a medical scan for procedures when the opaque patient marking indicia 38 will not be an obstruction to the medical professional.

The bottom surface 36 of the skin template substrate 30 contains a skin adhesive 40 that is stronger than the low strength adhesive 20 that bonds the two primary substrates together. The skin adhesive 40 must be stronger than the low strength adhesive 20 so that the skin template substrate 30 remains adhered to the patient's surface 84 while the imaging substrate 12 is being removed. The skin template substrate 12 consists of a the cut 120 in the middle. The die cut 120 provides open access for marking the patient's surface 84, while the perimeter area 122 remains adhered to the patient's surface 84.

A removable, protective backing sheet 50 is disposed over the skin adhesive 40. The backing sheet 50 protects the adhesive 40 when the aid 10 is not being used.

Anchor points 60 are placed in an asymmetrical pattern to prevent rotational errors during the realignment phase. According to one embodiment, a minimum of three anchor points 60 is needed to prevent rotational error. Alternatively, the shape of a singular anchor point 60 itself can be asymmetric. In a preferred embodiment, four anchor points 60 are placed on the aid, one near each corner. This allows for easy placement while applying each section of the aid to the body, as well as being easy to maintain. To create asymmetry, one of the anchor points 68 will be staggered and placed further away from its corner relative the distance placement of the other anchor points 62, 64, 66 from their respective corner. The anchor points 60 provide access to mark the patient's surface 84. There are a variety of forms the aforementioned anchor points system can take in either phase. In the first phase, the anchor points may be, but not limited to: holes 60 for skin demarcation; perimeter markings 200 delineating skin demarcation; ink stamp transfers; wet or dry temporary tattoo transfers; film-adhesive transfers; holes 60 for the marking of permanent tattoos; or holes 60 for the placement of sutures that act as anchor point markings. In the second phase, the anchor points 60 may be, but not limited to: holes 60 to visibly align over the skin markings; or perimeter markings 200 delineations to align with the skin markings. Holes for the placement and alignment of anchor points 60 reduce potential error compared to perimeter markings that may allow greater shifting.

In another preferred embodiment, FIG. 5 details a device that has been optimized for intraoperative use. The localizing aid 10 is comprised of similar layers as the preoperative use device above in FIG. 4. Anchor points 60 may not be needed, since the device is used intraoperatively during the procedure. There is no temporal delay that would require the removal and re-alignment of a second aid 10. In addition, the skin template substrate 30 in an intraoperative-type device may be a continuous incisable sheet as shown and described in FIG. 16. This incisable sheet may be made of polyethylene or polyurethane, and may have the added benefit of infection control. An antimicrobial coating, such as iodine, may be applied alongside the skin adhesive 40. By removing the top imaging substrate 12 after the intraoperative scan, the surgeon would have complete freedom to make multiple incisions with the skin image 32 pattern serving as a guide. However, a universal aid 10 may be desired for the sake of simplicity and inventory purposes of handling only a single SKU. Anchor points 60 may be included so that the aid 10 can be used in a preoperative nature if required, allowing it to be a universal device.

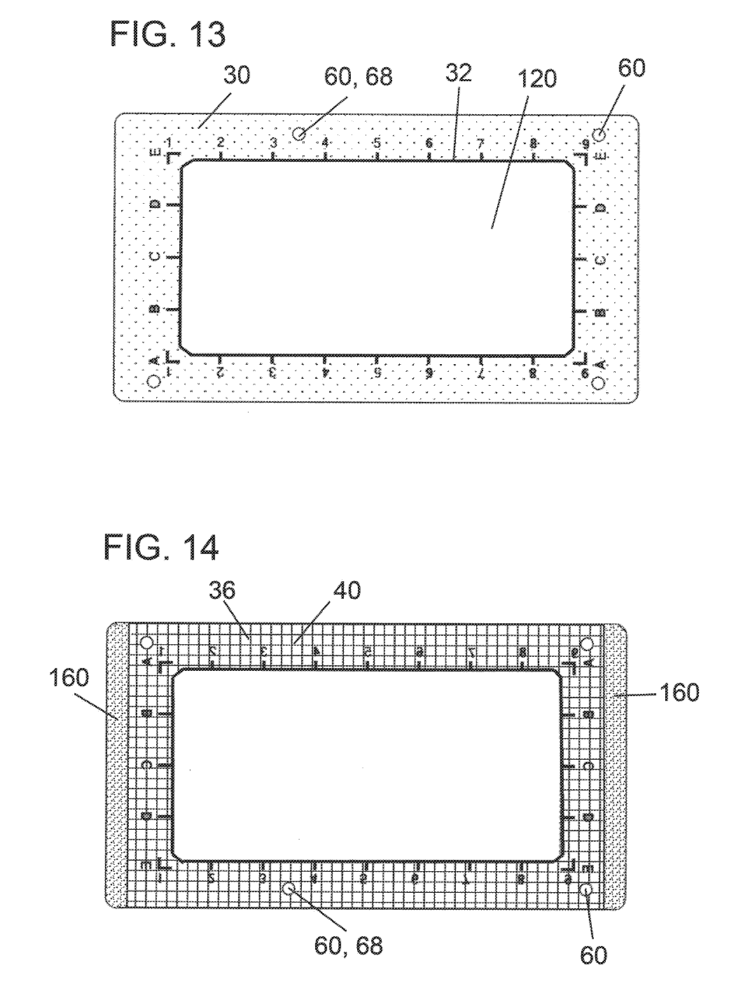

In addition, the preferred embodiment shown in FIG. 5 may utilize a skin template substrate 30 that is non-incisable, such as the sheets described in FIGS. 13 and 18. The skin template substrate 30 may utilize a die-cut to produce a single large cut-out 120 or a die-cut producing multiple small cut-outs 190. The localizing aid 10 would not require the anchor points 60 when used intraoperatively. However, as described above, anchor points 60 could be included if needed to allow for a universal device.

FIGS. 6 and 7 shows how the scan locational pattern 18 will be shown in an AP X-ray image. FIG. 6 is a schematic view of a medical image generated from a medical scanning procedure using the localization aid 10 as described herein. In FIG. 6, the lumbar portion of a patient's spine and the patient's hips are schematically shown as they might appear in an X-ray image. The resulting scan 102, clearly shows the opaque indicia 16, including in this instance intersecting grid lines 110 and both alpha and numeric characters 112. The scan image 102 also shows the internal anatomy 100 of the patient's body as shown, and in FIG. 5 a target area is identified with reference number 104. The target area 104 could be soft tissue, or in this case a portion of a vertebra located between the grid lines labeled C and D, and between the transverse grid lines 2 and 3.

FIG. 7 is an X-ray image of the upper torso region with the opaque locational reference pattern. The opaque material and the patient's bone appear as the same color. In this example, the X-ray setting shows opaque materials as white in color.

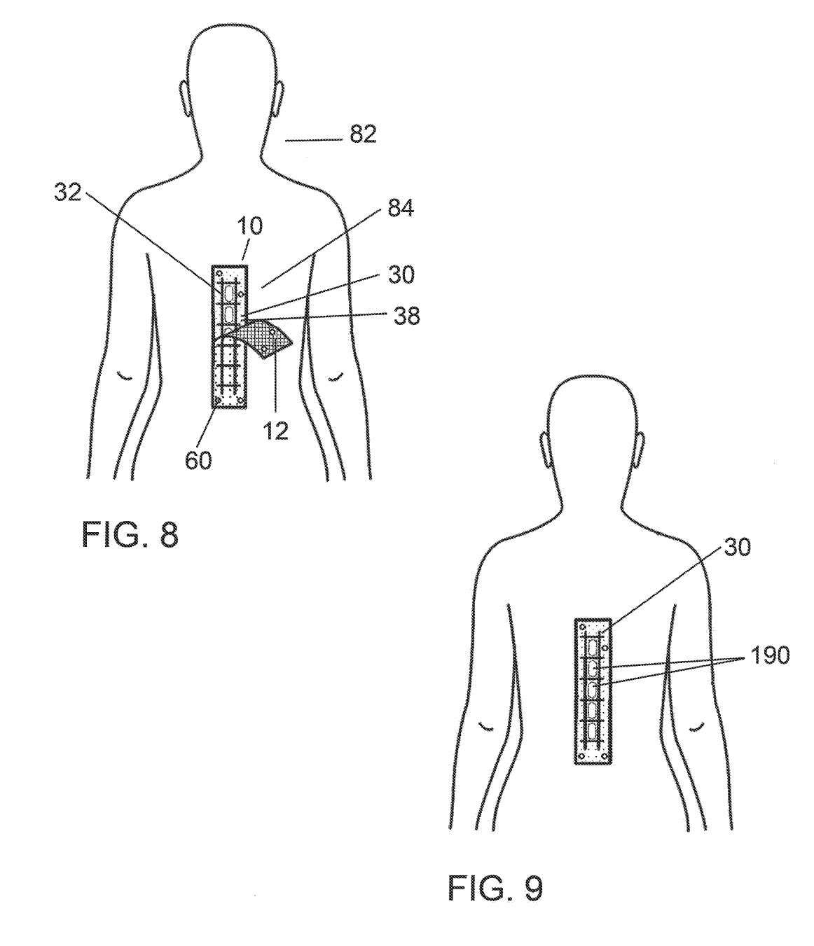

In FIG. 8 the localization aid 10 is schematically shown being removed from a patient's surface 84 to illustrate the skin template substrate 30 with patient marking indicia 38 transferred to the patient's surface 84. Thus, as the imaging substrate 30 is peeled upwardly and off the patient's surface 84, the skin template substrate 30 that was temporarily adhered to the bottom surface of the imaging substrate 12 has been transferred to the patient's surface 84 to provide a correlating skin image 32 with visual indicia.

In FIG. 9, the imaging substrate 12 has been removed and the skin template substrate 30 remains adhered to the patient's surface 84. The open cut-outs 190 allows for the medical professional to accurately mark the incision site 106 without removing the skin template substrate 30.

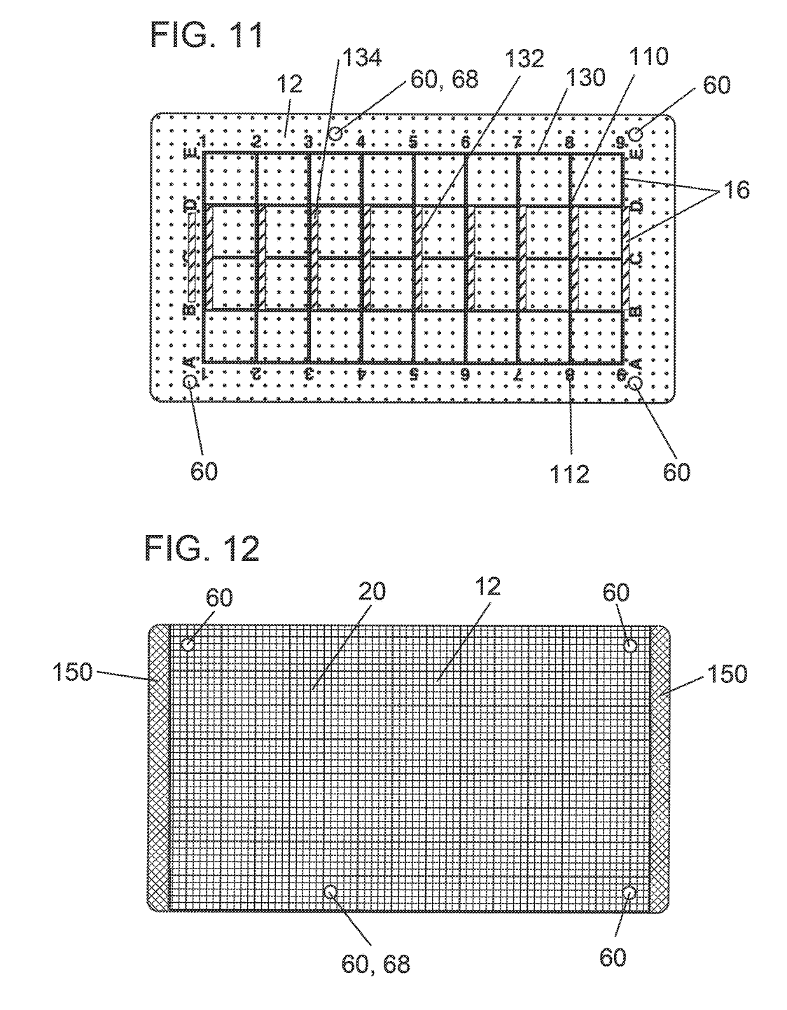

FIG. 11-FIG. 15 describes in detail each specific layer of the aid 10 in its preferred embodiments. FIG. 11 is a top view of the imaging substrate 12. The imaging substrate 12 needs to be relatively conformal to the skin during imaging, but rigid enough that it does not easily bend and stick upon itself. The material can be a polyester, PET or paper that can preferably range from 2-7 mils thick. The imaging substrate 12 provides support for the skin template substrate 30 to prevent it from flexing and adhering itself. The imaging substrate 12 should also be compatible with a heat curing process. The heat curing may be needed to dry the medical scan opaque ink that comprises the opaque indicia 16. The X-ray opaque material, or radiopaque material, can be, but are not limited to: barium sulphate, lead, tantalum, triphenylbismuth, tungsten or copper. These materials may form a paste, be suspended in an ink that cures, or die-cut. An MRI opaque material will be different from X-ray opaque materials. Two opaque materials may be needed so the device can be used in both X-rays and MR based imaging techniques. The MRI opaque material can be deposited on top of the X-radiation scan locational pattern 130 to form an MRI compatible pattern 132. The MRI opaque material may be a silicone-based polymer. The silicone-based polymer can be in a gel-like consistency that can be screen-printed or dispensed onto the top surface of the imaging substrate 12. The silicone-based polymer can be formed into a variety of different patterns, or molded into various shapes and sizes. To achieve ideal visualization on a lateral MRI image, the silicone-based polymer should be applied to the substrate with at least 2 mm of thickness so that its cross-section is clearly visible.

In one embodiment, the silicone-based polymer may be applied directly on top of the cured radiopaque scan locational pattern 130. The silicone-based polymer can be applied in a duplicate scan locational pattern as the radiopaque pattern 130. Alternatively, the imaging technique(s) of certain procedures may require that the silicone-based polymer to only partially duplicate the radiopaque locational pattern 130.

For example, imaging of the spine could require both a radiopaque pattern 130 for X-rays and an MRI compatible pattern 132 for MR imaging. The radiopaque pattern 130 could be printed in a traditional grid design comprised of intersecting horizontal and vertical lines 110 with alphanumeric label coordinates 112. The MRI compatible pattern 132 may only be dispensed in shorter horizontal lines 134 placed down the middle of the grid. The MRI compatible pattern 132 is not opaque to X-rays and will not appear on an X-ray image. In addition, the radiopaque pattern 130 will not appear on the MRI. This will prevent any unnecessary markings appearing on each image type.

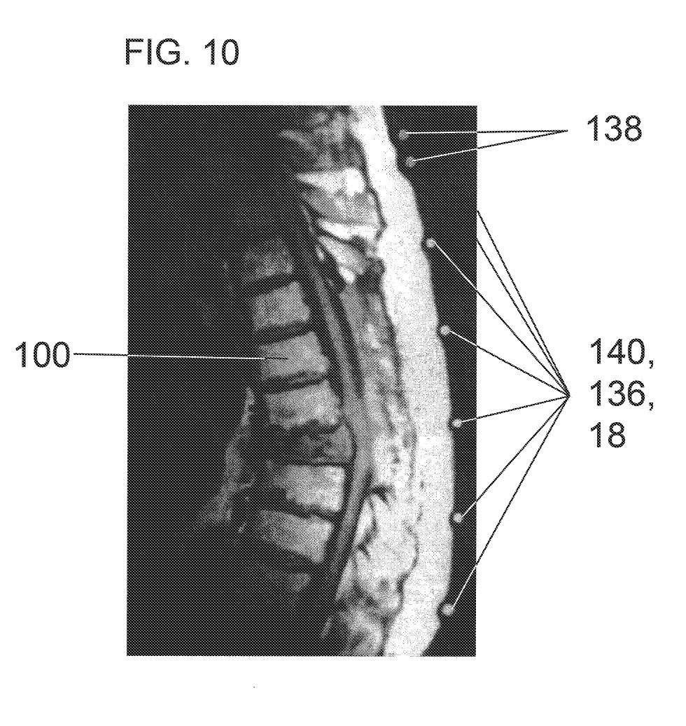

Typically, a spine MRI is taken in a lateral direction, which would result in the horizontal silicone-based polymer lines 134 appearing as a series of dots 136 above the vertebrae—a cross-section (see FIG. 10).

Alternatively, an AP X-ray image (See FIGS. 6 and 7) can be taken of the spine as well. The radiopaque pattern 130 with alphanumeric coordinates 112 will appear on the X-ray image overlapping the spine, chest and ribs. The medical professional can correlate the appropriate location in relation to its respective grid coordinate.

The lateral MRI would provide no alphanumeric coordinates. To compensate for the lack of index labels, the first set of horizontal lines could consist of two lines placed close together. The remaining horizontal lines are placed further apart, but at equal intervals. This distinction with the first set of horizontal lines creates a visual reference for the medical professional to determine where the starting set of lines is, since it would lack coordinate labels on the MRI. The purpose is to avoid counting and correlation errors due to mislabeling one of the horizontal lines as the incorrect starting point.

FIG. 10 is a lateral MRI view of a spine. The scan locational pattern 18 is viewed as a series of horizontal dots 136 due to the cross sectional view that the MRI is taken. The first two dots (i.e. the starting dots) 138 are located in closer proximity to each other relative to the spacing of the rest of the dots 140. This provides a clear starting point to reduce the risk of an incorrect count.

The distinction of a unique starting point is not limited to two lines placed closely together in relation to the other lines. The starting reference may also consist of a silicone-based polymer that is dispensed thicker or thinner than other lines. Overall, there must be a unique distinction in the pattern to identify a specific reference mark to allow for correct labeling of the pattern. The medical professional needs to be confident in correlating the target on the image to the correct reference mark on the patient without the risk of error due to mislabeling.

The silicone-based polymer can also be UV cured for quick and efficient drying. The cured silicone-based polymer will form into a soft solid with a supple, squishy property. A supple, gel-like property is beneficial to achieve opacity in an MRI. The soft, supple property of the silicone-based polymer does not mean it is delicate to handle though. The cured silicone-based polymer is durable enough to handle without tearing, breaking or flaking.

The soft, supple property is a result of a relatively low cross-linking density. However, a cross-linking density that is relatively too low will cause the silicone-based polymer to be too liquid and therefore cannot be dispensed into a desired pattern. It may also not cure into a desired solid. In addition, if the cross-linking density is relatively too high, the cured silicone-based polymer will not have a supple property and be too hard. The relatively harder silicone, with higher cross-linking, reduces MRI opacity. The silicone-based polymer can also adhere to most common substrates, such as plastics and metals.

Compared to other methods of creating opaque fiducial(s) on MRI, such as using liquid capsules (such as halibut oil or vitamin K) or tubes filled with a liquid solution, using a silicone-based polymer is easier and more efficient to manufacture; can be produced in various designs, shapes, thicknesses and molds; has a longer shelf life; can be produced quicker and on a larger scale; and is more cost efficient.

FIG. 12 is the bottom view of the imaging substrate 12. A low strength adhesive 20 is applied to the bottom surface of the imaging substrate 12. Along the top and bottom edges, a separation tab 150 is created by having non-adhesive strips exposed. The non-adhesive strips along the two edges create a separation tab 150 that allows for user-friendly separation between the imaging substrate 12 and the skin template substrate 30. If the low strength adhesive 20 covers the entire bottom surface of the imaging substrate 12, adhering non-adhesive strips to the imaging substrate's 12 edges can create the separation tab 150. Alternatively, the low strength adhesive 20 can be applied only to the middle section of the bottom surface of the imaging substrate, leaving the edges of the imaging substrate 12 non-sticky.

FIG. 13 is the top view of the skin template substrate 30. The skin template substrate 30 is a thin, conformal, breathable film material that is adhered to the patient's surface 84. A skin template locational reference pattern, or skin image 32, that correlates to the imaging substrate's scan locational pattern 18 is printed on the skin template substrate 30. The skin image pattern 32 is preferably non-opaque to medical imaging because its intended purpose is to provide visual reference on the skin for the medical professional. A die cut opening 120 in the middle of the skin template substrate 30 provides access to the patient's surface 84 to mark the site of the incision 106 or make the incision. The die cut 120 can be a single, large cut-out or divided into multiple quadrants 190 to provide easier guidance to line up the index markings.

FIG. 14 is the bottom view of the skin template substrate 30. A skin compatible adhesive 40 that is stronger than the low strength adhesive 20 of the imaging substrate 12 is applied to the bottom surface of the skin template substrate 30. Along the top and bottom edges of the bottom surface of the skin template substrate 30, a handling tab 160 is created by having strips of the protective backer 50 remain attached to the edges of the skin template substrate 30. The non-sticky strips along the two edges create a handling tab 160 that allows for user-friendly placement of the aid 10. By preventing the adhesive edges of the skin template substrate 30 from being exposed, the user can easily apply the aid 10 without the aid 10 sticking to their fingers. Once the aid 10 is applied to the patient's surface 84, the handling tabs 160 can be removed so the entire bottom surface of the skin template substrate 30 is adhered to the patient's surface 84. The handling tabs 160 can also remain attached to the skin template substrate 30 after adhesion to the patient 82 and remain as removal tabs to allow for easy removal from the patient's surface 84. Alternatively, the skin adhesive 40 can be applied only to the middle section of the bottom surface of the skin template substrate 30, leaving the edges non-sticky.

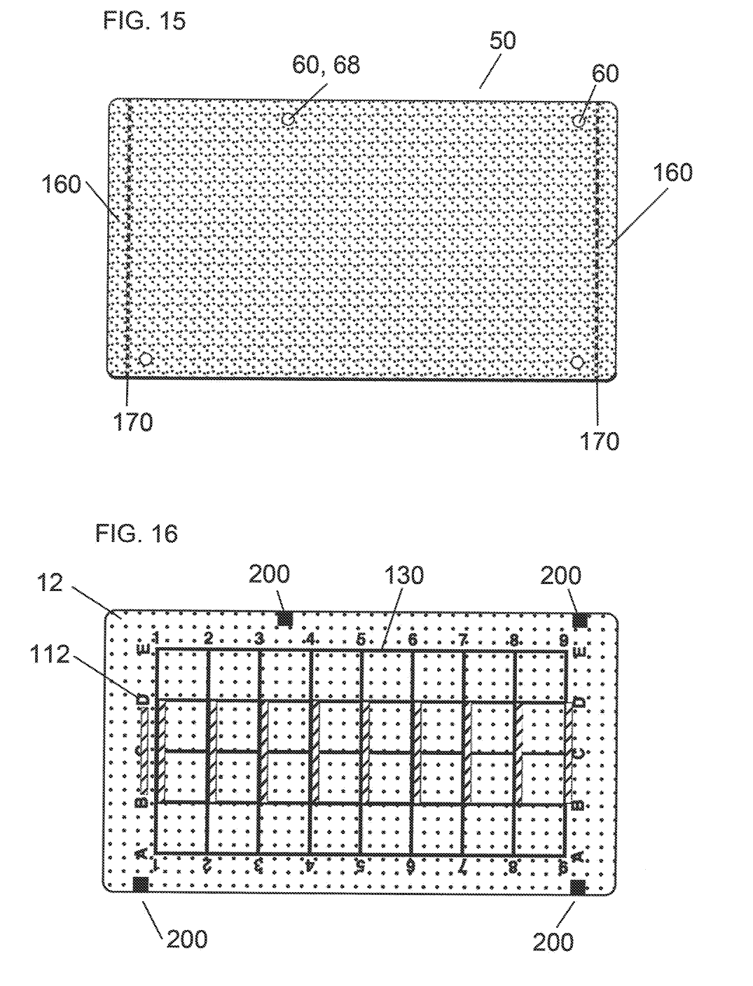

FIG. 15 is the bottom view of the removable protective backing sheet 50. A kiss cut 170 is placed along the top and bottom edges of the protective backing sheet 50. The kiss cut 170 is a cut that passes through the protective backing sheet 50, while the skin template substrate 30 and imaging substrate 12 remain intact. The kiss cut 170 creates a crack and peel label system which allows for the large section of the protective backing sheet 50 to be easily separated from the skin template substrate 30. Two thin strips of the protective backing sheet 50 remain attached to the aid 10 as handling tabs 160. The protective backing sheet 50 protects the skin adhesive 40 when the aid 10 is not being used.

FIG. 16 is the top view of another preferred embodiment of the imaging substrate 12. Instead of cutting anchor point holes 60 throughout the aid 10, visual anchor point markings 200 along the edge of the imaging substrate 12 are printed. The visual anchor point markings 200 allows for the user to mark the edges of the aid 10 to the patient's surface 84 as anchor point skin marks 70. During the re-alignment step, the edges of the anchor point markings 200 on the aid 10 are aligned to the edges of the skin marks 70 on the patient's surface 84.

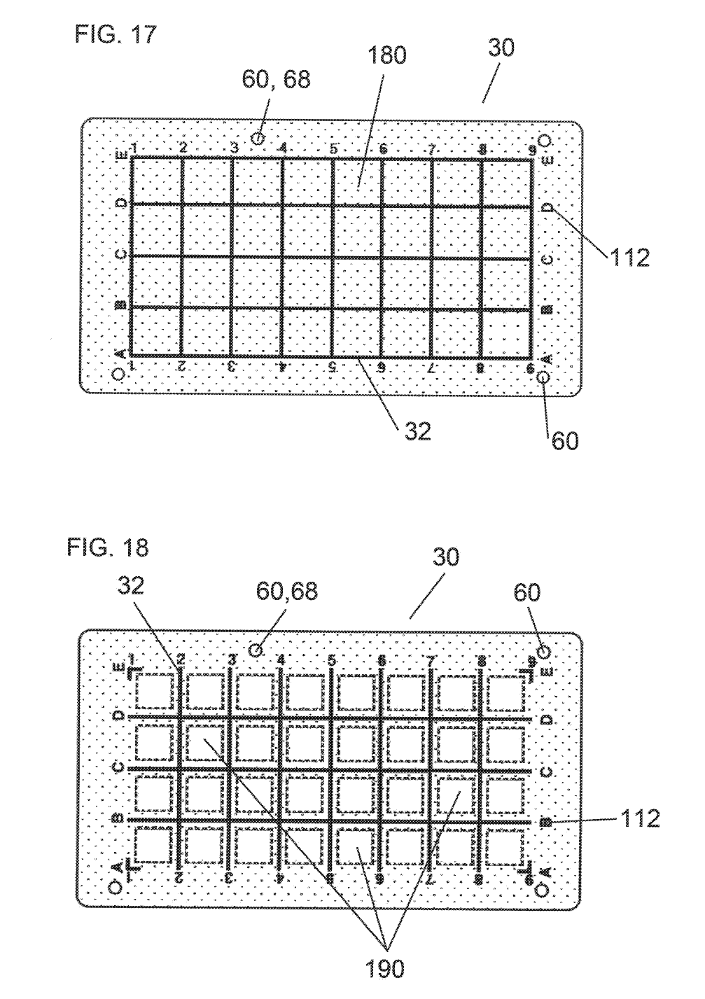

FIG. 17 is the top view of an alternative preferred embodiment of the skin template substrate 30. A continuous sheet 180 is utilized instead of a large die cut 120 being placed in the skin template substrate 30 as shown in FIG. 13. The continuous sheet 180 may be an incisable film material that the medical professional can cut through or perform some other invasive procedure through. The incisable film material will have the ability to not core when being cut through with a scalpel or surgical blade. The incisable film material can also contain an anti-microbial coating, such as iodine, along with the skin adhesive 40. The anti-microbial coating serves as infection control when placed on a patient's cleaned skin. An example material is 3M's Loban II™ or Smith and Nephew's (London, UK) OpSite Incise Drape™. The visible indicia printed on the continuous sheet 180 can be made from optimal, biocompatible ink, which would pose no added risk or potential obstruction to the surgeon. Moreover, subsequent intraoperative scans used to confirm internal processes would not be obstructed by the opaque indicia 16 on the imaging substrate 12 that was removed. If an incisable film material is used, the second phase of the aid would have to be performed intraoperatively after the patient's surface 84 is cleaned. If both phases occur during the operation with intraoperative imaging, the imaging substrate is an ideal support for the continuous sheet 180 with incisable properties. For example, after an initial fluoroscopic scan, it may be determined that the aid 10 was misplaced. The medical professional could simply peel off the aid 10 via the handling tabs 160 on the skin template substrate 30, with the top imaging substrate 12 acting as support for the extremely flexible incisable film material. The device could then be placed correctly for a second fluoroscopic scan.

Alternatively, the continuous film in FIG. 17 may be a porous membrane that the medical professional can use their pen 80 to the mark the patient's surface 84 through.

FIG. 18 is the top view of yet another alternative preferred embodiment of the skin template substrate 30. A die cut can be utilized to create multiple cut-outs 190 throughout the skin template substrate 30. The multiple cutouts 190 provide better visualization of the skin image 32 by allowing for continuous index lines to be visualized.

While the present invention has been described in terms of preferred and illustrated embodiments, it will be appreciated by those of ordinary skill that the spirit and scope of the invention is not limited to those embodiments, but extend to the various modifications and equivalents as defined in the appended claims.

1-14. (canceled)

15. A medical procedure localization aid, comprising:
a flexible multilayer sheet adapted for conformational application to a patient, said sheet comprising
an imaging substrate having reference indicia thereon comprising material opaque to medical imaging scans and arranged in a scan locational pattern, and
a skin template substrate having reference indicia thereon that registers at least partially with the reference indicia on the imaging substrate, the skin template removably bonded to a lower surface of the imaging substrate,
adhesive on a lower surface of the skin template and a removable backing sheet covering said adhesive; and
wherein said multilayer sheet may be adhered to a patient by removal of said backing sheet so that said lower surface of said skin template adheres to said patient, and said imaging substrate may subsequently be removed from said skin template with said skin template remaining adhered to said patient.

16. The medical procedure localization aid according to claim 15 further comprising plural patient marking holes formed through the multilayer sheet in an asymmetric pattern so that the location and orientation of said sheet may be marked on a patient.

17. The medical procedure localization aid according to claim 16 wherein the plural patient marking holes are formed through the flexible multilayer sheet around a periphery thereof.

18. The medical procedure localization aid according to claim 15 in which the flexible multilayer sheet defines a peripheral edge that is symmetric.

19. The medical procedure localization aid according to claim 15 wherein the skin template defines a sheet having at least one opening therein so that said patient's skin is exposed through said openings when imaging substrate is removed from said skin template.

20. The medical procedure localization aid according to claim 15 wherein the skin template defines a continuous sheet.

21. The medical procedure localization aid according to claim 20 wherein the skin template is incisable.

22. The medical procedure localization aid according to claim 21 wherein the skin template further comprises an anti-microbial coating.

23. The medical procedure localization aid according to claim 22 wherein the reference indicia further comprises a bio-compatible ink.

24. The medical procedure localization aid according to claim 15 further comprising adhesive applied to the lower surface of the imaging substrate to bond the skin template to said imaging substrate, and wherein the adhesive applied to the lower surface of the imaging substrate defines a lower strength adhesive relative to the adhesive applied to the lower surface of the skin template.

25. The medical procedure localization aid according to claim 19 further comprising adhesive applied to the lower surface of the imaging substrate to bond the skin template to said imaging substrate, and wherein the adhesive applied to the lower surface of the imaging substrate defines a lower strength adhesive relative to the adhesive applied to the lower surface of the skin template.

26. The medical procedure localization aid according to claim 25 wherein adhesive is applied only to select portions of the lower surface of the imaging substrate.

27. The medical procedure localization aid according to claim 26 wherein adhesive is not applied to the portions of the lower surface of the imaging substrate that define said at least one opening.

28. A medical procedure localization aid, comprising:
a flexible multilayer sheet defined by an upper imaging substrate having an upper surface and an opposed lower surface and reference indicia comprising material opaque to medical imaging scans and arranged in a scan locational pattern;
adhesive applied to at least select portions of the lower surface of the upper imaging substrate;
a skin template having an upper surface and an opposed lower surface, said skin template having reference indicia arranged in a scan locational pattern, the upper surface of the skin template adhered to the adhesive applied to the lower surface of the upper imaging substrate so that the scan locational pattern on the skin template registers at least partially with the scan locational pattern on the upper imaging substrate, wherein the upper imaging substrate is removable from the skin template;
adhesive applied to the lower surface of the skin template, said adhesive applied to the lower surface of the skin template relatively stronger than the adhesive applied to the lower surface of the upper imaging substrate; and
a removable protective backing sheet adhered to the adhesive applied to the lower surface of the skin template.

29. The medical procedure localization aid according to claim 28 further comprising sheet positioning means on said flexible multilayer sheet for marking the location and orientation of said sheet on a patient.

30. The medical procedure localization aid according to claim 29 wherein the sheet positioning means is further defined by at least one opening formed through said flexible multilayer sheet in an asymmetric pattern.

31. The medical procedure localization aid according to claim 30 in which the flexible multilayer sheet has a periphery that defines a symmetric sheet and wherein the sheet positioning means is further defined by at least 3 openings formed through said sheet adjacent the periphery thereof in an asymmetric pattern.

32. A medical procedure localization method for use in a medical procedure, comprising the steps of:
a) adhering to a patient in a desired location a multilayer sheet defined by an upper imaging substrate having reference indicia thereon comprising material opaque to medical imaging scans and arranged in a scan locational pattern, and a lower skin template substrate having reference indicia thereon that registers at least partially with the reference indicia on the upper imaging substrate;
b) creating a medical image of the patient, said medical image including the multilayer sheet;
c) removing the upper imaging substrate from the patient without removing the lower skin template; and
d) performing a medical procedure on the patient.

33. The medical procedure localization method according to claim 32 including, prior to step d), marking the position of the lower skin template on the patient by placing skin marks on the patient through plural asymmetrically arranged anchor points on said lower skin template, and subsequently removing said lower skin template.

34. The medical procedure localization method according to claim 33 including reapplying to the patient a second lower skin template having identical plural asymmetrically arranged anchor points onto the patient by aligning said plural asymmetrically arranged anchor points on said second lower skin template with said skin marks, and subsequently performing said medical procedure on said patient.
